(19) United States
(12) Patent Application Publication
(10) Pub. No.: US 2023/0220501 A1
(43) Pub. Date: Jul. 13, 2023

(54) PATHOGEN DETECTION SYSTEM

(71) Applicant: Whitworth University, Spokane, WA (US)

(72) Inventors: Philip S. Measor, Spokane, WA (US); Kent Jones, Spokane, WA (US)

(21) Appl. No.: 18/153,260

(22) Filed: Jan. 11, 2023

Related U.S. Application Data
(60) Provisional application No. 63/299,156, filed on Jan. 13, 2022.

(51) Int. Cl. C12Q 1/70; B01L 3/00; B01L 7/00

(52) U.S. Cl. CPC C12Q 1/701; B01L 3/502715; B01L 7/52; B01L 2300/0654; B01L 2300/1805; B01L 2300/1894; C12Q 1/6851

(57) ABSTRACT

Systems and a device for a pathogen detection system are described herein. For example, a pathogen detection system may include a pathogen detection device comprising an inlet, an outlet, and a reactive chamber; an imaging system comprising an excitation source and a fluorescence detection system; a substrate; and a heating element. In some examples, the pathogen detection system may be configured to detect one or more pathogens and/or viruses in a sample. For example, the pathogen detection device may receive a solution containing at least one of DNA or RNA and route the solution to the reactive chamber. Upon heating the solution, the pathogen detection device may further receive a probe containing one or more fluorescent dyes. The excitation source may excite the solution, and the detection system may detect an emission of the one or more fluorescent dyes.

### CROSS-REFERENCE TO RELATED APPLICATIONS

This application claims priority to and is a non-provisional application of U.S. Provisional Patent Application No. 63/299,156 filed on Jan. 13, 2022, the entire contents of which are incorporated herein by reference.

### REFERENCE TO SEQUENCE LISTING

The Sequence Listing associated with this application is provided in XML format in lieu of a paper copy and is hereby incorporated by reference into the specification. The name of the XML file containing the Sequence Listing is W058-0006US.xml. The text file is 158 KB, was created on Jan. 5, 2023, and is being submitted electronically via Patent Center.

### BACKGROUND

Polymerase Chain Reaction (PCR) systems by which users may detect viruses, or other pathogens, provides the general public with numerous benefits and opportunities. For example, PCR may allow users to effectively and accurately detect the presence of viruses in biological samples. However, most conventional PCR systems and devices are highly labor intensive and expensive and require a high degree of environmental control. One alternative to traditional PCR devices include microfluidic devices; however, these devices are typically expensive. Thus users may desire an alternative and more affordable PCR device.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an example pathogen detection system.

FIG. 2 illustrates a two-dimensional top-down view of an example embodiment of a pathogen detection device.

FIG. 3 illustrates a schematic diagram of a method of manufacturing a pathogen detection device.

FIG. 4 illustrates a schematic diagram of an alternate method of manufacturing a pathogen detection device.

FIG. 5A illustrates example conformations of a pathogen molecular probe designed to bind to the RNA encoding the spike protein of COVID.

FIG. 5B illustrates additional example conformations of a pathogen molecular probe designed to bind to the RNA encoding the spike protein of COVID.

FIG. 6 illustrates an example process for binding a molecular probe to the RNA or DNA target region that encodes a pathogen to detect the presence of the pathogen in a sample.

FIG. 7 illustrates a standard melting curve corresponding to binding a molecular probe to a RNA or DNA target.

FIG. 8 illustrates a proof of principle for a molecular probe designed to bind to a DNA target that replicates the RNA that encodes the spike protein of COVID.

FIG. 9A illustrates a 3D printed microwell design for a proof of principle demonstration of detecting a molecular probe.

FIG. 9B illustrates a zoomed in view of a portion of FIG. 9A around the dotted box.

### DETAILED DESCRIPTION

As discussed above, the current and conventional standard for detecting and diagnosing viruses and other pathogens is PCR. In order to detect viruses, the genomic deoxyribonucleic acid (DNA) or ribonucleic acid (RNA) of the virus is extracted from the virion capsid and a complimentary strand is multiplied many orders using a special polymerase until the DNA or RNA can be detected (e.g., multiplied about one million times). The detection is typically performed using DNA probes designed with a sequence specific to the virus strain desired to detect. Probes may include, for example, molecular beacons and/or intercalating dyes. The system used to detect the virus may typically include a desktop quantitative PCR (qPCR) instrument with optical excitation and/or detection and a controlled thermal cycler to activate the polymerase to copy. This process typically requires from about 30 to about 60 cycles of low (from about 20° Celsius (C) to about 50° C.) to high (from about 80° C. to about 90° C.) temperatures to amplify target DNA or RNA and detect an optical signal. Subsequently, a qPCR curve is typically generated, which may show the exponential growth of the complimentary DNA, if present, in the sample. The original amount of source DNA or RNA may be traced back to a cycle number that may be calibrated to a high level of sensitivity (e.g. down to 1 to 1000 genome copies/mL).

Due to the highly sensitive nature of PCR, an environment used to detect a virus must be highly controlled, requiring the use of various technologies such as HEPA filters, HVAC controls, laminar flow, and fume hoods. Additionally, qPCR is performed in centralized labs by highly trained personnel. In order to reduce the risk of contamination, some qPCR methods rely on instruments that may automate preparation of samples and detection of viruses or other pathogens. These automated qPCR methods are developed for testing at centralized laboratory facilities for high-throughput and fast turn-around times. These times typically vary from about 24 hours to 14 days. However, due to the nature of viruses which are being detected, patients from which samples are being tested may be placed in quarantine while awaiting results in order to prevent further spread of the virus. In addition to the role in community health, these long quarantine times force patients to enter isolation, and may result in severe psychological effects, such as depression. Additionally, long quarantine times may result in lost productivity, such as time missed from work. Thus, fast turn-around times are critical. Quickly receiving a negative result may allow a patient to return to socialization and the workplace, increasing happiness and productivity. Alternatively, quickly receiving a positive result may allow for appropriate contact tracing, retesting, and treatment, effectively circumventing the spread of infection and epidemic progression.

Point-of-care (POC) pathogen detection instruments are one method in which patients may receive fast results. POC diagnostics is a healthcare approach that may enable the detection of viruses, or other pathogens, at a place a patient receives healthcare, such as a doctor's office, rather than sending a sample to a lab. Current PCR automated systems may include POC devices located in hospital settings. For example, some POC PCR devices may rely on cartridge systems. Such systems place samples in cartridges, which are then placed in a thermal cycler. In some examples, an optical detection system may be used to detect a pathogen. These cartridges are typically constructed from plastic and may contain reagents necessary to perform specific tests for detecting viruses or other pathogens. However, these cartridges are bulky and may be expensive to manufacture.

One solution to current POC PCR devices presents itself in the form of lab-on-a-chip (LOC) devices. LOC devices can perform tests that are normally performed in a lab but miniaturizes such tests on a small chip. This provides advantages such as high-throughput and automated processing. LOC devices typically include microfluidic channels to transport samples in a fluid for testing. Using LOC devices for pathogen detection presents many advantages, such as reduced sample volumes, reagents, and waste footprints. However, LOC devices are typically extremely costly to fabricate. For example, conventional LOC devices are manufactured using silicon microfabrication techniques which require cleanrooms and highly trained operators.

A solution presents itself in the form of three-dimensional (3D) printing. 3D Printing involves the creation of 3D formations using computer aided design (CAD) structures with materials that are added, joined, or solidified (as opposed to removing from bulk material) using various methods. Thus, LOC devices may be manufactured using 3D printing. Not only is the cost of fabricating a LOC device substantially less expensive than conventional techniques, but 3D printing eliminates the need for a cleanroom, a myriad of instruments, and trained professionals.

### Pathogen Detection System

This application describes a 3D printed LOC pathogen detection system. For example, the system for pathogen detection may include a LOC device. The device may include, in some examples, an inlet, an outlet, and/or a reactive chamber. The system may further include an imaging system, which may include an excitation source and/or a fluorescence detection component. Additionally, the system may include a substrate and/or a heating element.

In some examples, the pathogen detection system may be configured to detect one or more viruses or other pathogens. While this disclosure describes the system as configured to detect SARS-CoV-2, the system may be used to detect one or more of any viruses or pathogens, including variants associated with SARS-CoV-2. For example, the system may receive, at the inlet of the pathogen detection device, a solution containing a biological sample. For example, the system may receive at least one of DNA or RNA associated with a virus or pathogen that the system is to detect. The solution may include, for example, a buffer solution. The system may then route the solution to the reactive chamber of the pathogen detection device for processing and detection.

In some examples, the system may heat, via the heating element, the solution to amplify the DNA or RNA. For instance, in the example that the solution contains DNA, the heating element may be used to activate a DNA amplification process. Various amplification processes may be used, such as PCR, Loop-Mediated Isothermal Amplification (LAMP), Nucleic Acid Sequence Based Amplification (NASBA), Self-Sustained Sequence Replication (3SR), Rolling Circle Amplification (RC), and Ligase Chain Reaction (LCR), to name a few examples. In some examples, the pathogen detection device containing the solution may be placed directly onto of the heating element. The heating element may include, for example, a Peltier device, a heating block, a thermal electric cooling device, a hot plate, and/or a resistive heater.

The system may then receive, at the inlet of the pathogen detection system, at least one of a DNA or RNA probe. In some examples, the probe may contain one or more fluorescence dyes. Although the use of a probe is described, various other reporting mechanisms may be used, such as intercalating dyes or molecular beacons.

In some examples, the system may then excite, via the excitation source of the imaging system, the solution. The excitation source may contain an optical imaging system including a sequence of lenses and mirrors to shape and direct the optical excitation. In response to exciting the solution, the system may detect, via the fluorescence detection system, an emission of the one or more fluorescence dyes. For example, the system may rely on various optical responses when detecting the emission of the one or more fluorescent dyes, such as Raman scattering and Rayleigh scattering. The system may additionally rely on various detection devices, such as an Electron Multiplying Charge-Coupled Device (EMCCD), a Photomultiplier Tube (PMT), a Charge-Coupled Device (CCD), a Single-Photon Avalanche Diode (SPAD), and an optical power meter, to name a few examples.

In some examples, the LOC device may be manufactured using 3D printing methods. For example, the LOC may be manufactured using stereolithography (SLA) 3D printing, in which light may be focused onto a vat of undeveloped, light-curable resin. The curable resin may increase in absorption as the wavelength of light is shortened, therefore decreasing the penetration depth of light. The decreased penetration depth may decrease the amount of resin that is polymerized, yielding a more controlled z-axis for the channels. The resin may also have an optical absorption characterized for about approximately 300 nanometers (nm) to approximately 1000 nm. The light source may include a digital light processing (DLP) projector with a LED light source between approximately 350 nm and approximately 380 nm, but not limited to these wavelengths. The light source may include a laser with, but not limited to, a raster-scanned light spot using a motorized mirror mount.

To manufacture the pathogen detection device disclosed herein, for example, a build platform may be lowered into the vat of resin. Upon lowering the build platform into the vat, a light source may be formed in a specified pattern. In some examples, the pattern may be pre-programmed by the user and/or another device. The light may project through the vat of resin onto the build platform such that the undeveloped resin that comes into contact with the light is solidified to form a single layer of developed resin on the build platform. In some examples, the build platform may lower one layer to recoat with new undeveloped resin, and the process may be repeated until the design is complete. The light source may be programed to emit different patterns such that each layer of developed resin consists of a different pattern. In some examples, the patterns may repeat themselves. Once the process is complete, the build platform may be raised from the vat of undeveloped resin, and any remaining undeveloped resin may be removed from the completed 3D pathogen detection device.

In examples, the pathogen detection device may be manufactured using a series of, but not limited to, patterns to create layers of developed resin. For example, once the build platform may be lowered into a vat of undeveloped resin, the light source may emit a first pattern such that a first layer of resin may be deposited on the build platform, the first layer being developed resin. Additionally, the light source may emit light in a second pattern such that the second layer of resin is deposited on the first layer of resin. The second pattern may include a first and second portion of developed resin and a third portion of undeveloped resin, where the third portion may be deposited between the first and second portion of the second layer. As such, the second layer of resin may begin to form the outer wall of the pathogen detection device, wherein the outer wall may consist of developed resin.

In some examples, the light source may emit a third pattern such that a third layer of resin is deposited on the second layer of resin. The third layer of resin may include a first portion, second portion, and third portion that may be developed resin. Further, the third layer may include a fourth portion and fifth portion of resin that may be undeveloped resin. In examples, the fourth portion of the third layer of resin may be deposited between the first portion and second portion of the third layer, and the fifth portion of the third layer may be deposited between the second portion and third portion of the third layer of resin. By doing so, the third layer may make up a portion of the pathogen detection device, such that at least a portion of the exterior walls and at least a portion of one or more interior walls. In some examples, this may form a portion of an outer resin cladding, wherein the outer resin cladding may be comprised of undeveloped resin and encompassed by developed resin.

In some examples, the light source may emit a fourth pattern such that a fourth layer of resin is deposited on the third layer of resin. The fourth layer of resin may include a first portion, second portion, third portion, and/or fourth portion that may be developed resin. Further, the fourth layer may include fifth portion, sixth portion, and/or seventh portion of resin that may be undeveloped resin. By doing so, the fourth layer may build upon the third layer, elongating the exterior and/or interior walls of the outer cladding and defining at least a portion of an inner resin cladding, wherein the inner resin cladding may be deposited within the outer resin cladding. In some examples, the inner resin cladding may be comprised of undeveloped resin and may be encompassed by the developed resin.

In some examples, the light source may emit a light in the third pattern such that a fifth layer of resin may be deposited on the fourth layer of resin. The fifth portion of the fourth layer of resin may then be deposited between the first portion and second portion of the fourth layer, the sixth portion of the fourth layer may be deposited between the second portion and third portion of the fourth layer of resin, and the seventh portion of the fourth layer of resin may be deposited between the third portion and fourth portion of the fourth layer. As a result, a fifth layer may build upon the fourth layer, further elongating the exterior and/or interior walls of the outer cladding and fully forming an inner resin cladding encompassed by developed resin.

In some examples, the light source may emit light in the second pattern such that a sixth layer of resin may be deposited on the fifth layer of resin. By deposing the sixth layer, the exterior and/or interior walls of the outer cladding are further elongated. Additionally, the light source may emit light in the first pattern such that a seventh layer of resin may be deposited on the sixth layer of resin. By doing so, the pathogen detection device may be fully formed, wherein an inner resin cladding may be deposited within an outer resin cladding.

To provide support to internal structures, the user and/or another device may program the light source to create temporary support structures between layers and/or between portions of layers, which may be removed later in manufacturing.

In other examples, manufacture of the pathogen detection device may include a “bottom up” approach using inverted stereolithography. This process may require the manufacturing process to start by lowering the built platform to touch the bottom of the vat of resin. The light source may emit a first pattern upward through the vat of resin to come into contact with the build platform, thus solidifying and developing undeveloped resin which the light may come into contact with. In some examples, the vat may be tilted at a pre-defined angle, up to 5 degrees by way of example, and peeled away from the now-hardened developed resin, in which the developed resin may detach from the bottom of the vat while remaining attached to the build platform. New liquid undeveloped resin may flow in the newly created space between the developed resin and bottom of the vat, and the process may be repeated until the pathogen detection device is complete.

In some examples, the pathogen detection device may go through a surface roughness treatment, that may be called microfluidic curing. After manufacturing the pathogen detection device using 3D printing, residual non-polymerized undeveloped resin may exist within the channels of the device. To remove the undeveloped resin, the channels may be patterned to connect to a microfluidic connector and eject the remaining undeveloped resin, leaving at least a layer of undeveloped resin. The remaining resin may be exposed with a UVA lamp post-ejection to be polymerized.

### Pathogen Probe

In addition to systems and methods for pathogen detection, this application describes a pathogen probe designed to be used in the pathogen detection system (described above) for detecting viruses, such as the virus SARS-CoV-2. For example, a pathogen probe was designed specific to the spike protein of SARS-CoV-2. This probe was designed in-silco using the National Center for Biotechnology Information's (NCBI) protein and DNA-sequence database which contained currently known SARS-CoV-2 strains (as of October 2021). A specific conserved region of the genome (the region responsible for encoding the spike protein) was selected, and a 36 base hairpin-shaped pathogen probe was designed to denature at about 60° C.:

(SEQ ID NO: 1)
5′-GCGGGCGAAAAGTAGATCTTCAATAAATGAGCCCGC-3′.

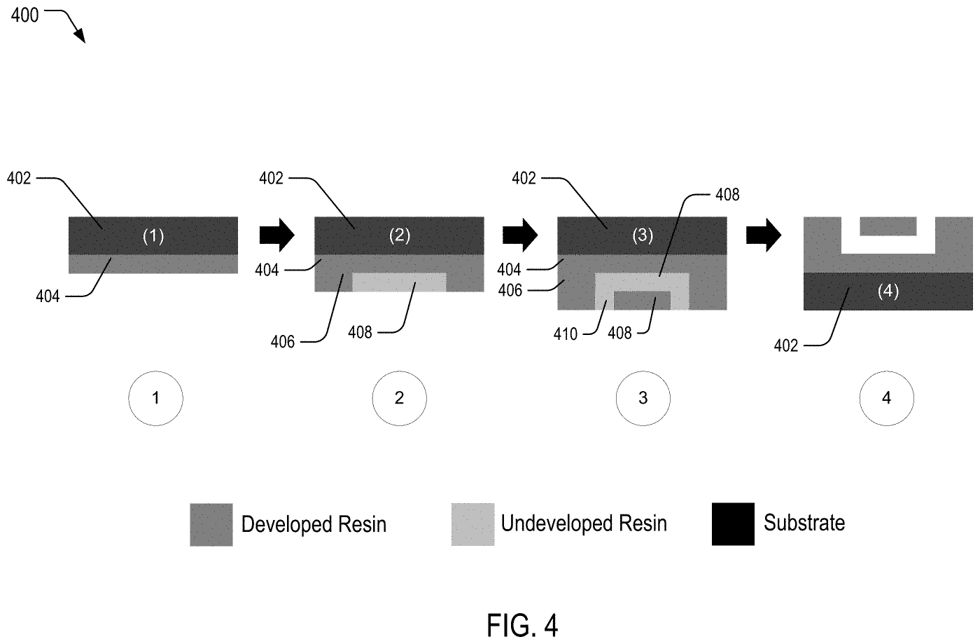

Simulations of the likely RNA sequence resulted in five likely confirmations, as described below with respect to FIG. 4. The SARS-CoV-2 specific pathogen probe was then ordered from Integrated DNA Technologies, Inc., with a corresponding red dye (TYE665), a quencher, and a 100 base oligonucleotide corresponding to a SARS-CoV-2 genome:

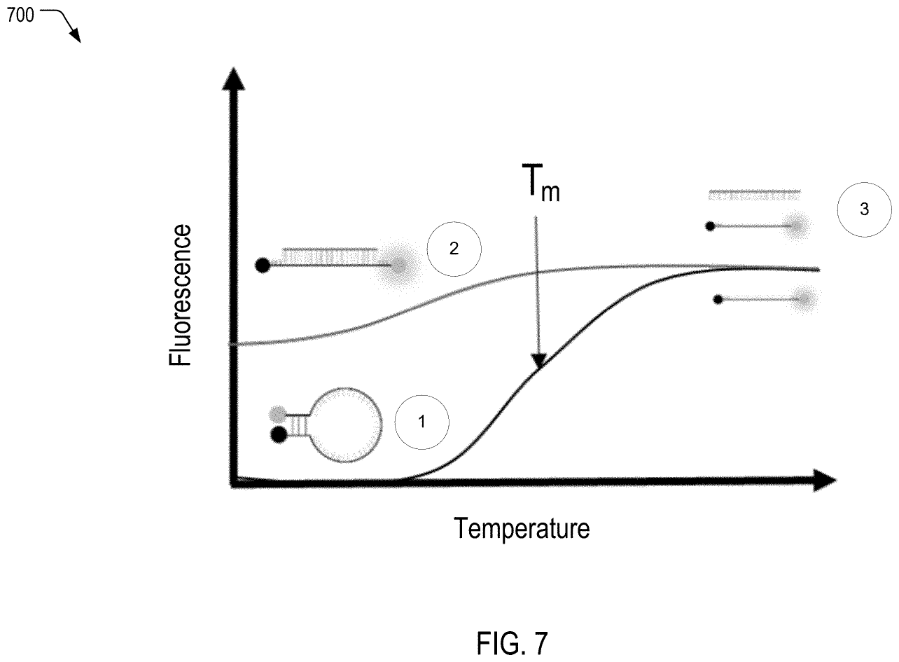

(SEQ. ID NO: 2)
5′-ACAAATATTACCAGATCCATCAAAACCAAGCAAGAGG
TCATTTATTGAAGATCTACTTTTCAACAAAGTGACACTTG
CAGATGCTGGCTCATCAAACAA-3′

where the underlined portion is complementary to the pathogen probe. A melting curve was obtained to confirm the probe functionality using a thermal cycler. Proof-of-principle experiments were performed by 3D printing a microarray with reaction chambers of 1,166 microwells with dimensions of about 116 μm by about 116 μm, as described below with respect to FIG. 7.

### Example Embodiments for Pathogen Detection System

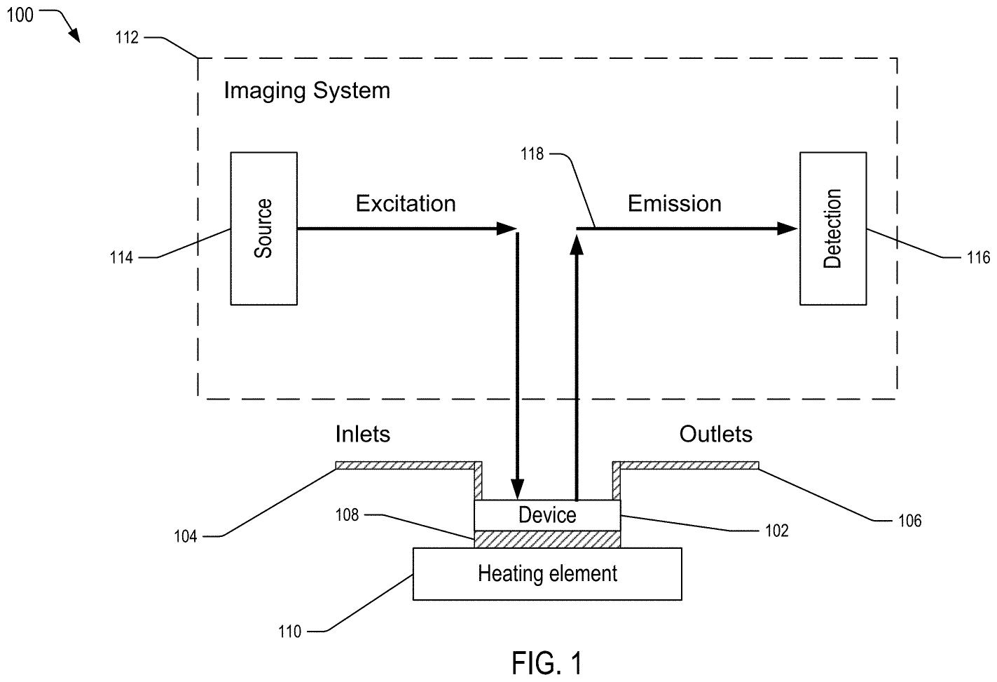

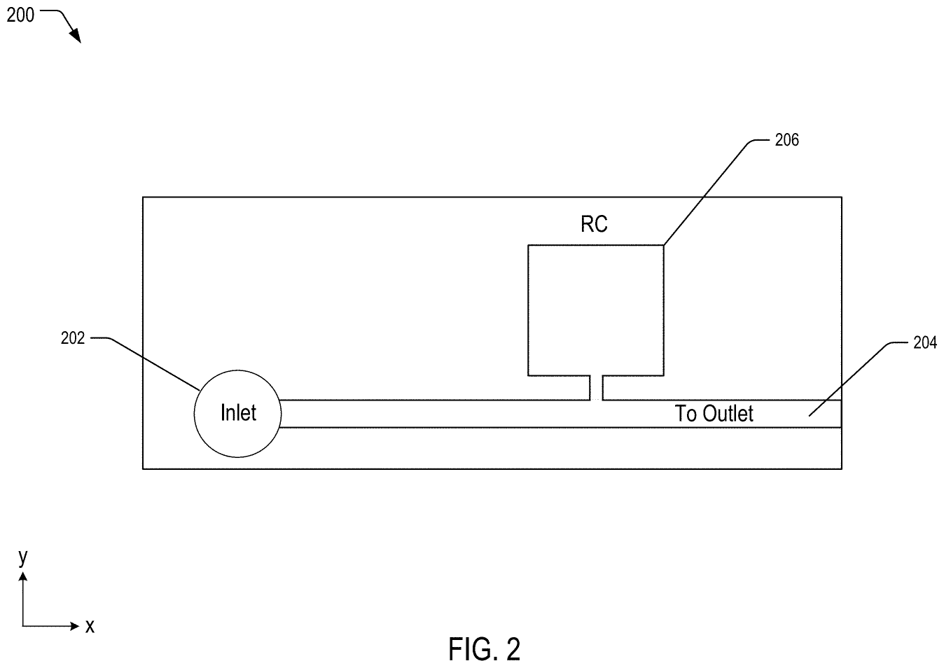

FIG. 1-FIG. 4 illustrate example embodiments for detecting pathogens using a pathogen detection system and pathogen detection device. For example, FIG. 1 illustrates an example pathogen detection system 100. The pathogen detection system 100 may include a pathogen detection device 102, which is described below with respect to FIG. 2. The pathogen detection device 102 may include an inlet 104, an outlet 106, and a reactive chamber (not pictured in the current embodiment). In some examples, the pathogen detection system 100 may be configured to detect one or more pathogens or viruses. For example, the system may receive, at the inlet of the pathogen detection device 102, a solution. The solution may contain at least one of DNA and/or RNA of one or more viruses and/or pathogens the pathogen detection system 100 may detect. The DNA or RNA may be, in some examples, a product that may be the compliment of one or more pathogens, such as streptococcus, Staphylococcus aureus, methicillin-resistant Staphylococcus aureus (MRSA), coliform bacteria, Escherichia coli (E. Coli), Salmonella, Shigella, Clostridium difficile (C. diff), tuberculosis, anthrax, mycoplasma, chlamydophila, legionella, Bordetella pertussis, Clostridium tetani, Borrelia burgdorferi, Borrelia mayonii, Clostridium botulinum, Vibrio cholera, Neisseria meningitides, Haemophilus influenza, Listeria monocytogenes, Treponema pallidum, gardnerelaa vaginalis, Neisseria gonorrhoeae, encodes Candida albicans, lactobacillus, microsporum canis, tinea pedis, Candida, Aspergillus, Histoplasma, Cryptococcus neoformans (C. neoformans), Blastomyces, Coccidioides, protozoa, helminths, ectoparasites that cause diseases such as malaria, toxoplasmosis, trichomoniasis, giardiasis, tapeworm, roundworm, lice, scabies, leishmaniasis, or river blindness, to name a few non-limiting examples.

In some examples, the solution may also include a buffer solution that may consist of TE buffer, TAE buffer, TBE buffer, tris(hydroxymethyl)aminomethane (tris), ethylenediaminetetraacetic acid (EDTA), boric acid, acetic acid, HCl, NaOH, NaCl, MgCl, cetyltrimethyl ammonium bromide (CTAB), polyvinylpyrrolidone (PVP), isopropanol, ethanol, RNase, DNase, potassium acetate, ultrapure water, water (H₂O) or NH₄Cl. In some examples, the pathogen detection device 102 may be coupled to a substrate that may consist of silicon, glass slides, fused silica, quartz, sapphire, gallium nitride, germanium, Gorilla Glass, indium tin oxide, lithium tantalite, silicon nitride, silicon germanium, silicon carbide, epitaxial silicon, silicon-on-insulator (SOI), single-crystal quartz, aluminum oxide, Coming Eagle Glass, borosilicate, BK7 glass, soda lime glass, thermal oxide, zinc oxide, II-V semiconductors, II-VI semiconductors, solar cells, charged-coupled devices, liquid crystal displays to name a few 108.

In some examples, the pathogen detection device 102 may be coupled to a heating element 110. The heating element may include a Peltier device, a heating block, a thermal electric cooling device, a hot plate, and/or a resistive heater, to name a few non-limiting examples. In some examples, applying heat to the pathogen detection device 102 may initiate amplification of the DNA and/or RNA contained in the solution. Various amplification processes may be used, such as PCR, Loop-Mediated Isothermal Amplification (LAMP), Nucleic Acid Sequence Based Amplification (NASBA), Self-Sustained Sequence Replication (3SR), Rolling Circle Amplification (RC), and Ligase Chain Reaction (LCR) to name a few examples.

In some examples, the pathogen detection device 102 may receive, at the inlet 104 of the pathogen detection device 102, at least one of a DNA and/or RNA probe, which is described below with respect to FIGS. 4-6. In response to receiving the probe, the pathogen detection system 100 may then excite the solution containing the DNA and/or RNA and probe via an excitation source 114. The excitation source may contain an optical imaging system including a sequence of lenses and mirrors to shape and direct the optical excitation.

In response to exciting the solution containing the DNA and/or RNA and probe, the pathogen detection system 100 may then detect, via a detection system 116, an emission 118 of the one or more fluorescence dyes. For example, the pathogen detection system 100 may rely on various optical responses when detecting the emission of the one or more fluorescent dyes, such as Raman Scattering and Rayleigh Scattering. The system may additionally rely on various detection devices, such as an Electron Multiplying Charge-Coupled Device (EMCCD), a Photomultiplier Tube (PMT), a Charge-Coupled Device (CCD), a Single-Photon Avalanche Diode (SPAD), and an optical power meter, to name a few examples.

FIG. 2 illustrates a two-dimensional top-down view of an example pathogen detection device 200. As described above with respect to FIG. 1 and similar to the pathogen detection device 102, the pathogen detection device 200 may be implemented in a pathogen detection system, such as pathogen detection system 100. For example, the pathogen detection device 200 may include an inlet 202, an outlet 204, and/or a reactive chamber 206. In some examples, an input sample, such a buffer solution containing DNA and/or RNA, may be introduced to the pathogen detection device 200 via the inlet 202. The solution may then be routed to the reactive chamber 206 for processing and detection. By way of example, in the case of DNA detection, a virus genomic DNA may be mixed with reagents and may be routed to the reactive chamber 206 for DNA amplification.

In some examples, the inlet 202 and/or the outlet 204 may be connected using a syringe and/or other pump via microfluidic tubing which may transfer the sample from one reactive chamber to another, and introduce reagents, fluids, and/or gasses, to name a few non-limiting examples.

While the current embodiment depicts a simple inlet 202, outlet 204, and reactive chamber 206, the pathogen detection device 200 may include any number of inlets 202, outlets 204, and reactive chambers 206 may be used to multiplex sample tests for one or more samples of DNA and/or RNA corresponding to detectable pathogens and/or viruses. In the case of multiple reactive chambers 206, the same or different reagent(s) may be introduced to the inlet 202 depending on the pathogen and/or virus being tested.

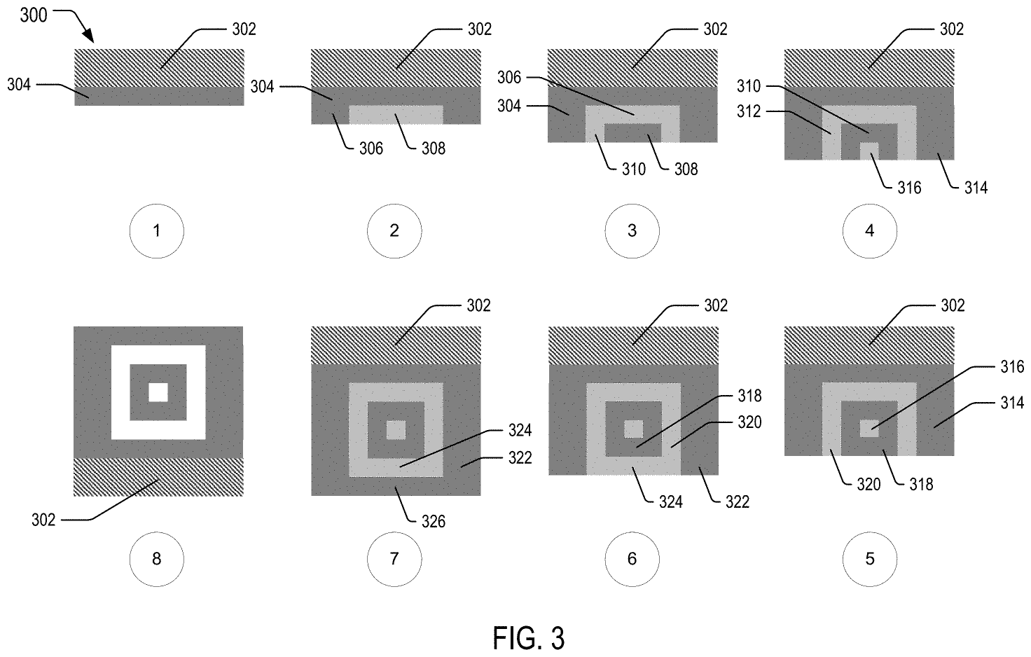

FIG. 3 and FIG. 4 illustrate schematic diagrams of methods of manufacturing a pathogen detection device, which may be the same or similar to that described with respect to FIG. 1 and FIG. 2. It should be understood that while the operations described with respect to FIG. 3 and FIG. 4 are described in stepwise processes, the processes may be performed in a different order and/or in parallel and may include more or less steps than that illustrated.

FIG. 3 illustrates a schematic diagram 300 of a method of manufacturing a pathogen detection device. In examples, the pathogen detection device may be manufactured using a series of patterns to create layers of developed resin. For example, the pathogen detection device may be manufactured using SLA 3D printing using, but not limited to, a DLP projector to emit light in a user specified pattern.

At step 1, a build platform 302 may be lowered into a vat of, but not limited to, undeveloped, UV-curable resin. The resin maybe fabricated from a formula comprising of monomer poly(ethylene glycol) diacrylate (PEGDA) as a base, and may be cured with photoinitiator bis(2,4,6-trimethylbenzoyl)-phenylphosphineoxide (Irgacure 819). In some examples, an additive UV absorber may be added to optimize microfluidic channel z-height detail. The UV-absorber may contain, but not limited to, avobenzone. In some examples, the resin may be sensitive to a light source of a 3D printer, such as about 365 nm light. This sensitivity may, upon printing the pathogen detection device, render the resin transparent to visible light, allowing light to move through the pathogen detection device. In some examples, the resin may be multi-layered. The layers may include, for example, microfluidic channels, mixers, optical waveguides, heaters, filters, photo-detectors, chemical detectors reaction chambers, electrical sources, conductors to name a few.

In some examples, a light source may emit one or more lasers at specified wavelength(s) in a first pattern of light directed toward the build platform 302. Upon contact with the undeveloped resin, the undeveloped resin may polymerize to form a first layer of developed resin 304 deposited upon the build platform 302. In examples, the pattern of light may be pre-programed and determined by a user and/or another machine.

At step 2, the light source may emit a light in a second pattern directed toward the build platform 302 and the first layer of developed resin 304 such that a second layer of resin may be deposited on the first layer of developed resin 304. In some examples, the second pattern may emit light so that specified portions of the undeveloped resin come in contact with the light, polymerizing the undeveloped resin to create developed resin, while other portions of the undeveloped resin remain undeveloped. For example, the light source may emit light in a second pattern such that the second layer of resin may include portions of developed resin 306 while a second portion may remain undeveloped resin 308, the second layer of undeveloped resin 306 deposited between the portions of the second layer of developed resin 308.

At step 3, the light source may emit light in a third pattern such that a third layer of resin is deposited on the second layer of resin, including the second layer of developed resin 304 and second layer of undeveloped resin 308. Similar to step 2, the third pattern may include portions of developed resin 310 and portions of undeveloped resin 312, the portions of the third layer of undeveloped resin 312 deposited between the third layer of developed resin 310.

At step 4, the light source may emit light in a fourth pattern such that a fourth layer of resin is deposited on the third layer of resin, including the third layer of developed resin 310 and third layer of undeveloped resin 312. Similar to step 2 and 3, the fourth pattern may include portions of developed resin 314 and portions of undeveloped resin 316, the portions of the fourth layer of undeveloped resin 316 deposited between the portions of the fourth layer of developed resin 314.

At step 5, the light source may emit light in the third pattern, similar to that in step 3, such that a fifth layer of resin is deposited on the fourth layer of resin, including the fourth layer of developed resin 314 and fourth layer of undeveloped resin 316. Similar to steps 2, 3, and 4, the fifth pattern may include portions of developed resin 318 and undeveloped resin 320, the portions of the fifth layer of undeveloped resin 320 deposited between the portions of the fifth layer of developed resin 318.

At step 6, the light source may emit light in the second pattern, similar to that in step 2, such that a sixth layer of resin is deposited on the fifth layer of resin, including the fifth layer of developed resin 318 and fifth layer of undeveloped resin 320. Similar to steps 2, 3, 4, and 5, the sixth pattern may include portions of developed resin 322 and undeveloped resin 324, the portion of the sixth layer of undeveloped resin 324 deposited between the portions of the sixth layer of developed resin 322.

At step 7, the light source may emit light in the first pattern, similar to step 1, such that a seventh layer of resin is deposited on the sixth layer of resin, including the sixth layer of developed resin 322 and sixth layer of undeveloped resin 324. Similar to step 1, the seventh layer may include a portion of developed resin 326.

At step 8, the pathogen detection device may be inverted, and the undeveloped resin may be ejected.

FIG. 4 illustrates a schematic diagram 400 of an alternate method of manufacturing a pathogen detection device. The method illustrated in FIG. 4 may be the same or similar to that illustrated in FIG. 3, above. For example, at step 1, a build platform 402 may be lowered into a vat of undeveloped, UV-curable resin, to name a non-limiting example. In some examples, the resin may be sensitive to a light source of a 3D printer, such as a 365 nm light. In some examples, the light source may emit one or more lasers at specified wavelength(s) in a first pattern of light directed toward the build platform 402. Upon contact with the undeveloped resin, the undeveloped resin may polymerize to form a first layer of developed resin 404.

At step 2, the light source may emit a light in a second pattern directed toward the build platform 402 and the first layer of developed resin 404 such that a second layer of resin may be deposited on the first layer of developed resin 404. In some examples, the second pattern may emit light so that specified portions of the undeveloped resin come in contact with the light, polymerizing the undeveloped resin to create developed resin, while other portions of the undeveloped resin remain undeveloped. For example, the light source may emit light in a second pattern such that the second layer of resin may include portions of developed resin 406 while a second portion may remain undeveloped resin 408, the second layer of undeveloped resin 406 deposited between the portions of the second layer of developed resin 408.

At step 3, the light source may emit light in a third pattern such that a third layer of resin is deposited on the second layer of resin, including the second layer of developed resin 404 and second layer of undeveloped resin 408. Similar to step 2, the third pattern may include portions of developed resin 410 and portions of undeveloped resin 412, the portions of the third layer of undeveloped resin 412 deposited between the third layer of developed resin 410.

At step 4, the pathogen detection device may be inverted, and the undeveloped resin ejected.

### Example Embodiments for Pathogen Probe

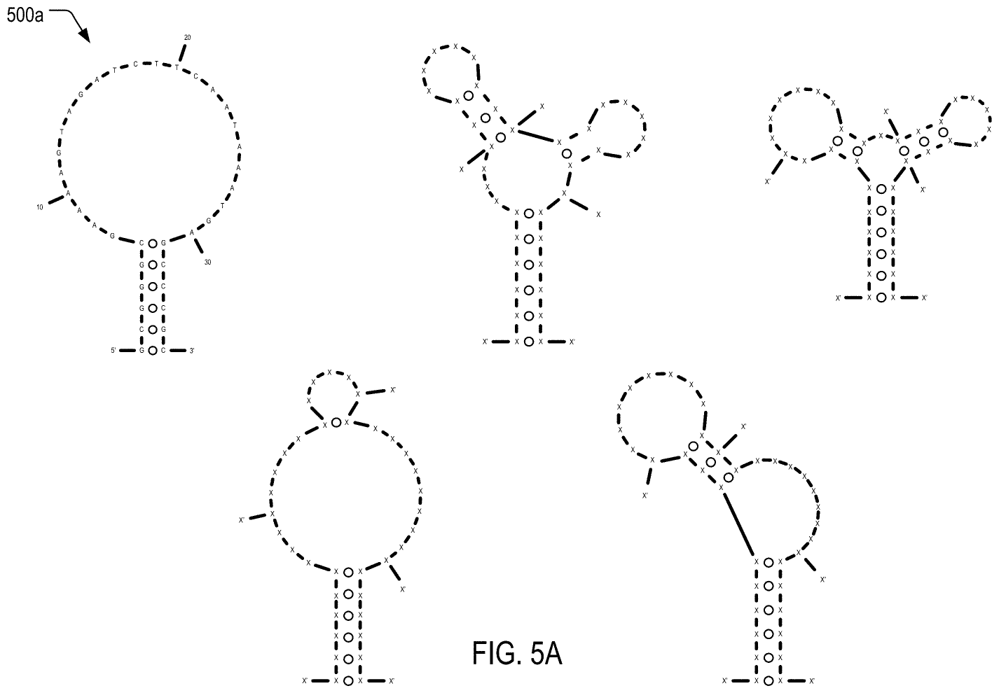

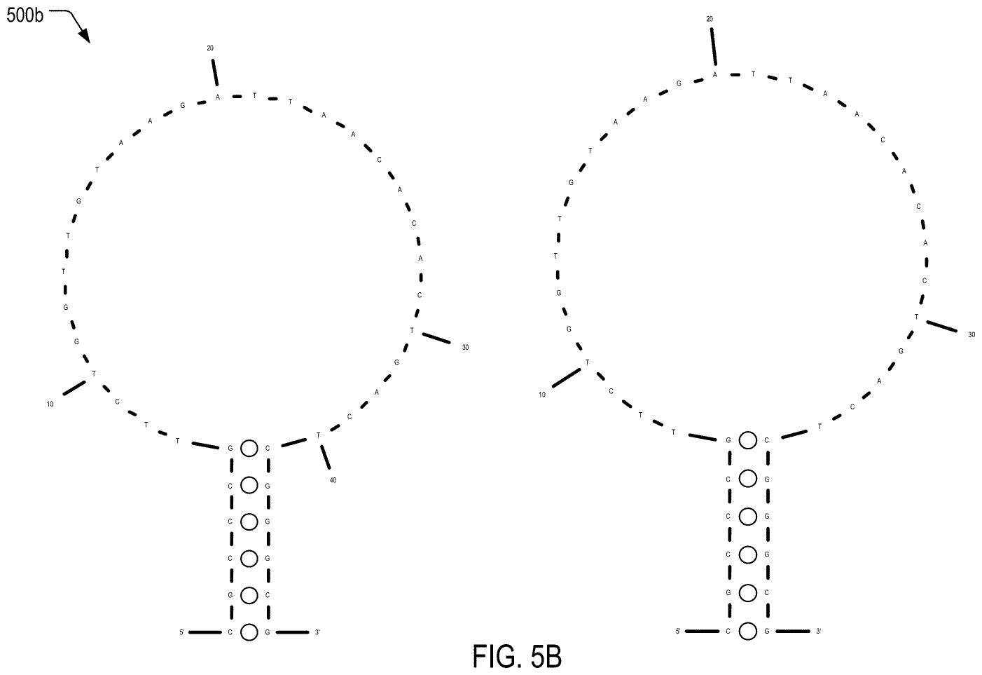

FIGS. 5A and 5B illustrates example conformations 500a and 500b of a pathogen probe, depicted in the current embodiment as a hairpin probe, designed to the spike protein of SARS-CoV-2. For example, the pathogen probe was designed using the NCBI's protein and DNA sequence database which contained the known SARS-CoV-2 strains at the time. A specific conserved region of the genome was selected, and a 36 base pathogen probe was designed to denature at about 60° C.:

(SEQ ID NO: 1)
5′-GCGGGCGAAAAGTAGATCTTCAATAAATGAGCCCGC-3′.

Possible derivatives of the probe are, for example but not limited to,

(SEQ ID NO: 3)
5′-GCGGGCTCATTTATTGAAGATCTACTTTTCGCCCGC-3′,
(SEQ ID NO: 4)
5′-CGCCCGCTTTTCATCTAGAAGTTATTTACTCGGGCG-3′,
(SEQ ID NO: 5)
5′-GCGGGCGAAAAGUAGAUCUUCAAUAAAUGAGCCCGC-3′,
(SEQ ID NO: 6)
5′-GCGGGCUCAUUUAUUGAAGAUCUACUUUUCGCCCGC-3′,
(SEQ ID NO: 7)
5′-CGCCCGCUUUUCAUCUAGAAGUUAUUUACUCGGGCG-3′.

Simulations of the likely RNA sequence resulted in five likely conformations 400 with the highest melting temperature of about 63.4° C. In some examples, the pathogen probe may include a corresponding dye, such as red dye (TYE665), a quencher, and/or a 100 base oligonucleotide corresponding to a SARS-CoV-2 genome:

(SEQ. ID NO: 2)
5′-ACAAATATTACCAGATCCATCAAAACCAAGCAAGAGG
TCATTTATTGAAGATCTACTTTTCAACAAAGTGACACTTG
CAGATGCTGGCTCATCAAACAA-3′

where the underlined portion is complementary to the pathogen probe.

Alternatively, a different segment of the SARS-CoV2 genome was selected 5′-AGUCAGUGUGUUAAUCUUACAACCAGAACUCAAUUACCCCCUGCAUACACUAA UUCUUUCACACGUGGUGUUUAUUACCCUGACA-3′ (SEQ ID NO: 15) or its DNA equivalent 5′-AGTCAGTGTGTTAATCTTACAACCAGAACTCAATTACCCCCTGCATACACTAATT CTTTCACACGTGGTGTTTATTACCCTGACA-3′ (SEQ ID NO: 16). The DNA equivalent, reverse complement of the target SARS-CoV2 genome sequences is 5′-TGTCAGGGTAATAAACACCACGTGTGAAAGAATTAGTGTATGCAGGGGGTAATT GAGTTCTGGTTGTAAGATTAACACACTGACT-3′ (SEQ ID NO: 27).

These correspond to the following primers:

Forward primer:
(SEQ ID NO: 17)
5′-AGTCAGTGTGTTAATCTTACAACC-3′
and
Reverse primer:
(SEQ ID NO: 18)
5′-TGTCAGGGTAATAAACACCACG-3′.

The reverse complements of these primers are:

Forward primer:
(SEQ ID NO: 19)
5′-GGTTGTAAGATTAACACACTGACT-3′
and
Reverse primer:
(SEQ ID NO: 20)
5′-CGTGGTGTTTATTACCCTGACA-3′.

From the sequence of the genome in SEQ ID NOs: 15 and 16, various pathogen probes were designed. Possible probes designs include, for example, but not limited to,

(SEQ ID NO: 21)
5′-GCGCCCTGTCAGGGTAATAAACACCACGTGTGA
AAGAATTGGGCGC-3′,
(SEQ ID NO: 22)
5′-TGCAGGGGGTAA-3′,
(SEQ ID NO: 23)
5′-CGCCCGTTCTGGTTGTAAGATTAACACACTGACTCGGGCG-3′,
(SEQ ID NO: 24)
5′-GCGCCCAATTCTTTCACACGTGGTGTTTATTACCCTGACAGG
GCGC-3′,
(SEQ ID NO: 25)
5′-TTACCCCCTGCA-3′,
(SEQ ID NO: 26)
5′-CGCCCGAGTCAGTGTGTTAATCTTACAACCAGAACGGGCG-3′,
(SEQ ID NO: 28)
5′-GCGCCCUGUCAGGGUAAUAAACACCACGUGUGAAAGAA
UUGGGCGC-3′,
(SEQ ID NO: 29)
5′-UGCAGGGGGUAA-3′,
(SEQ ID NO: 30)
5′-CGCCCGUUCUGGUUGUAAGAUUAACACACUGACUCGGGCG-3′,
(SEQ ID NO: 31)
5′-CGCGGGACAGUCCCAUUAUUUGUGGUGCACACUUUCUUAA
CCCGCG-3′,
(SEQ ID NO: 32)
5′-UUACCCCCUGCA-3′,
and
(SEQ ID NO: 33)
5′-CGCCCGAGUCAGUGUGUUAAUCUUACAACCAGAACGGGCG-3′.

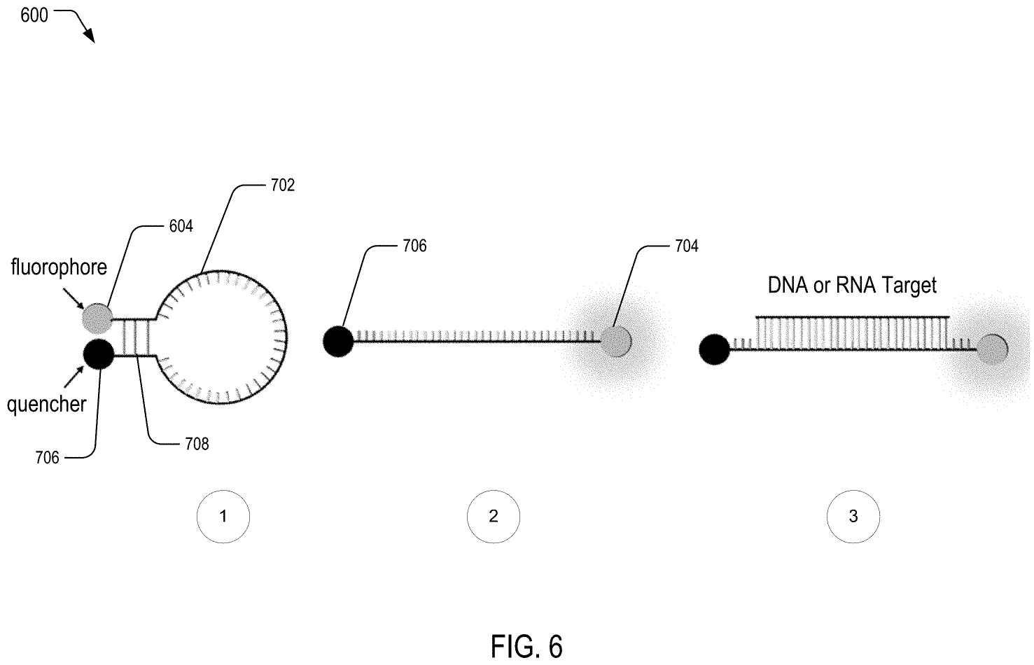

FIG. 6 illustrates an example process 600 for binding a probe to the spike protein of SARS-CoV-2 to detect the presence of the virus in a sample. The process 600 may be performed in a pathogen detection device in a pathogen detection system, as described above and with respect to FIGS. 1-4. For example, step one (indicated by “1”) illustrates a pathogen probe 502 designed to bind to a sike protein of SARS-CoV-2. The probe 602 may contain a fluorophore 604, which may be a type of fluorescent probe which may fluoresce when illumined by a specific wavelength of light. The probe may also contain a quencher 606, which may absorb light of a certain frequency and/or color range. In some examples, one or more portions of a stem 608 of the pathogen probe 602 may be complimentary, allowing the quencher to bind to the fluorophore.

Step two (indicated by “2”) illustrates the probe 602 after the probe has been exposed to heat, such as the heating element 106 described in FIG. 1. For example, in response to applying heat to a sample containing the probe 602, the probe 602 may become unbound, and the fluorophore 604 and the quencher 606 may unlink from one another. In some examples, the fluorophore 604 and the quencher 606 may be a distance apart from one another such that the fluorophore may fluoresce.

At step three (indicated by “3”), complementary DNA (cDNA) of SARS-CoV-2 may bind to the probe 602. For example, if the sample contains SARS-CoV-2 cDNA, the cDNA may preferentially bind to the probe 602. In some examples, the temperature, which was raised at step two, may be lowered. Probes 602 which have bound to cDNA will be prevented from re-folding and may produce fluorescence. However, unbound probes 602 may re-fold, similar to that depicted in step one. Thus, the more target DNA is present in a sample, the more fluorescence may be detected from the sample.

FIG. 7 illustrates a standard melting curve corresponding to the process illustrated in FIG. 6. For example, as indicated by “1,” a probe may be bound at a first temperature. However, in response to raising the temperature past a melting point 702, the probe may be unbound, as indicated by “2.” In some examples, a sample containing the probe may contain complimentary DNA, which may bind to the unbound probe, resulting in fluorescence. However, in some examples, as indicated by “3,” the sample may not contain complimentary DNA, leaving the probe unbound to complimentary DNA. In response to lowering the temperature, the unbound probes may re-bind to itself, resulting in little to no fluorescence.

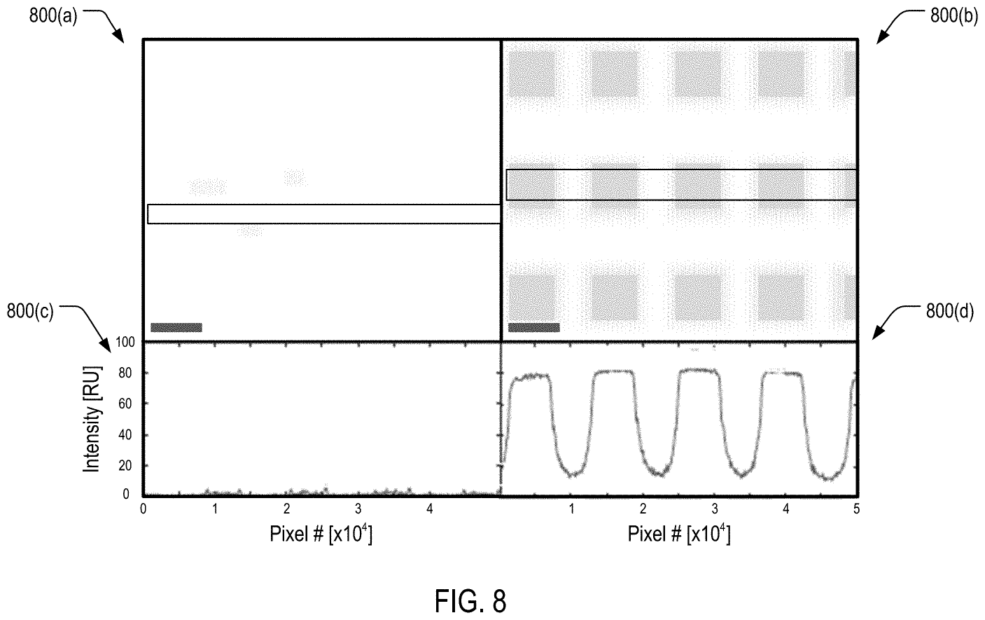

FIG. 8 illustrates a proof of principle for a probe. For example, to produce images 800(a) and 800(b) and corresponding intensity plots 800(c) and 800(d), probes without complimentary target DNA were introduced into a qPCR thermal cycler and ran through five cycles, ramping the temperature form about 25° C. to about 90° C., holding the temperature for about 20 seconds, and ramping the temperature back to down to about 25° C. Reagents with various conditions were then introduced and probed using the qPCR to obtain melting curves. In some examples, when the probes themselves were introduced to the qPCR by themselves, the fluorescence was low, indicating no target DNA present. Similarly, when the probes were mixed with random plasmid DNA and introduced to the qPCR cycles, the fluorescence was also low, indicating there was no target DNA present. However, when the designed probes mixed with complimentary synthetic target DNA were introduced, the probes bound to the complimentary DNA, producing a high fluorescence, thus indicating the presence of target DNA.

These reagent combinations were introduced onto a 3D printed microwell array device, such as a pathogen detection device described in FIGS. 1-4 and imaged with a confocal microscope system using orange excitation and collecting red fluorescence bandpass filters. 800a Illustrates an image of fluoresce imaged using a confocal microscope in which probes and negative plasmin random DNA was present. As illustrated, no fluorescence was detected. Alternatively, as illustrated in 800b, probes and complimentary target DNA were introduced, resulting in a high level of fluorescence, indicating a positive and specific detection of target DNA strand (synthetic SARS-CoV-2 nucleic acid).

The intensity was extracted from a portion of the images 800(a) and 800(b), and plotted in 800(c) and 800(d), respectfully. As depicted, the intensity is 2±1 relative units for the plasmid DNA sample illustrated in 800(b). However, when there was target DNA present the intensity was 80±1, resulting in a signal-to-noise ratio (SNR) of ˜7 for the positive sample, suggesting that a limit-of-detection (LOD) of less than ˜300 nM is possible.

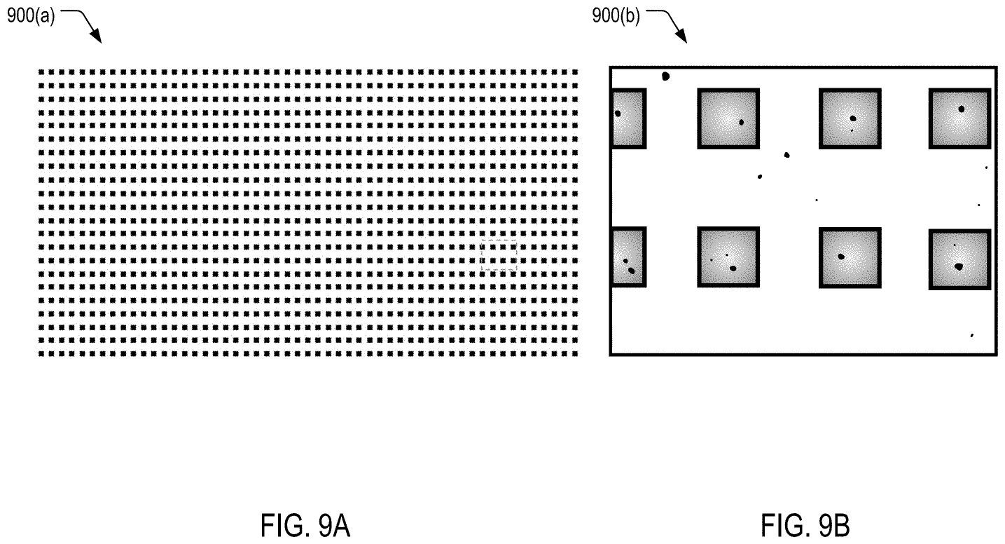

FIGS. 9A and 9B illustrate an alternate embodiment of a proof of principle for a probe. FIG. 9A illustrates an example 3D printed microwell design 900(a) of an example pathogen detection device. For example, similar to that described with respect to FIG. 8., proof-of principle experiments were performed by 3D printing a microarray reaction chambers of 1,166 microwells with about 116 μm by about 116 μm dimensions. FIG. 9B illustrates an example brightfield microscope image 900(b) of a 3D printed microwell, similar to that depicted in FIG. 9A. For example, FIG. 9B illustrates an image indicating the presence of target DNA within the pathogen detection device. For instance, when the designed probes mixed with complimentary synthetic target DNA were introduced to the pathogen detection device, the probes bound to the complimentary DNA, producing a high fluorescence, thus indicating the presence of target DNA.

Variants of the sequences disclosed and referenced herein are also included. Variants of the nucleic acid sequences of the probe disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the nucleic acid sequences disclosed herein.

“% sequence identity” refers to a relationship between two or more sequences, as determined by comparing the sequences. In the art, “identity” also means the degree of sequence relatedness between nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of two binding molecules with an affinity or Ka (i.e., an equilibrium association constant of a particular binding interaction with units of 1/M) equal to or greater than 10⁵M⁻¹, while not significantly associating with any other molecules or components in a relevant environment sample. “Specifically binds” is also referred to as “binds” herein. Binding may be classified as “high affinity” or “low affinity”. In particular embodiments, “high affinity” refer to binding with a Ka of at least 10⁷M⁻¹, at least 10⁸ M⁻¹ at least 10⁹ M⁻¹ at least 10¹⁰ M⁻¹, at least 10¹¹ M⁻¹ at least 10¹² M⁻¹ or at least 10¹³ M⁻¹. In particular embodiments, “low affinity” refers to binding with a Ka of up to 10⁷M⁻¹, up to 10⁶M⁻¹, up to 10⁵M⁻¹. A variety of assays are known for detecting binding as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, aspects of the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

RNA encoding viral proteins can be derived from adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses. In particular embodiments, RNA encoding viral proteins are derived from SARS-COV-2, CMV, EBV, flu viruses, hepatitis A, B, or C, herpes simplex, HIV, influenza, Japanese encephalitis, measles, polio, rabies, respiratory syncytial, rubella, smallpox, varicella zoster, West Nile, and/or Zika.

CMV proteins include envelope glycoprotein B and CMV pp65; EBV proteins include EBV EBNAI, EBV P18, and EBV P23; hepatitis proteins include the S, M, and L proteins of hepatitis B virus, the pre-S antigen of hepatitis B virus, HBCAG DELTA, HBV HBE, hepatitis C viral RNA, HCV NS3 and HCV NS4; herpes simplex proteins include immediate early proteins and glycoprotein D; human immunodeficiency virus (HIV) proteins include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; human papillomavirus virus (HPV) viral antigens include the L1 protein; influenza proteins include hemagglutinin and neuraminidase; Japanese encephalitis proteins include proteins E, M-E, M-E-NS1, NS1, NS1-NS2A and 80% E; malaria proteins include the Plasmodium proteins circumsporozoite (CSP), glutamate dehydrogenase, lactate dehydrogenase, and fructose-bisphosphate aldolase; measles proteins include the measles virus fusion protein; rabies proteins include rabies glycoprotein and rabies nucleoprotein; respiratory syncytial proteins include the RSV fusion protein and the M2 protein; rotaviral proteins include VP7sc; rubella proteins include proteins E1 and E2; varicella zoster proteins include gpl and gpll; and zika proteins include pre-membrane, envelope (E), Domain III of the E protein, and non-structural proteins 1-5.

Additional particular exemplary viral proteins sequences include:

| Source | Sequence |
|---|---|
| Nef | VGFPVTPQVPLRPMTYKAAVDLSHFLKEKGGL |
| (66-97): | (SEQ ID NO: 8) |
| Nef | HTQGYFPDWQNYTPGPGVRYPLTFGWLYKL |
| (116-145) | (SEQ ID NO: 9) |
| Gag p17 | EKIRLRPGGKKKYKLKHIV |
| (17-35) | (SEQ ID NO: 10) |
| Gag p17-p24 | NPPIPVGEIYKRWIILGLNKIVRMYSPTSILD |
| (253-284) | (SEQ ID NO: 11) |
| Pol 325-355 | AIFQSSMTKILEPFRKQNPDIVIYQYMDDLY |
| (RT 158-188) | (SEQ ID NO: 12) |
| CSP central | NANPNANPNANPNANPNANP |
| repeat region | (SEQ ID NO: 13) |
| E protein | AFTFTKIPAETLHTVTEVQYAGTDGPCKVPA |
| Domain III | QMAVDMQTLTPVGRLITANPVITEGTENSKM |
| | MLELDPPFGDSYIVIGVGE |
| | (SEQ ID NO: 14) |

See Fundamental Virology, Second Edition, eds. Fields, B. N. and Knipe, D. M. (Raven Press, New York, 1991) for additional examples of viral antigens.

### CONCLUSION

Although the discussion above sets forth example implementations of the described device and techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A system for pathogen detection comprising:
a pathogen detection device comprising:
an inlet;
an outlet; and
a reactive chamber;
an imaging system comprising:
an excitation source; and
a fluorescence detection system;
a substrate; and
a heating element;
wherein the pathogen detection system is configured to:
receive, at the inlet of the pathogen detection device, a solution containing at least one of DNA or RNA;
route the solution to the reactive chamber of the pathogen detection device;
heat, via the heating element, the solution;
receive, at the inlet of the pathogen detection device, at least one of a DNA or RNA probe, the probe containing one or more fluorescence dyes;
excite, via the excitation source of the imaging system, the solution and the probe; and
detect, via the fluorescence detection system, an emission of the one or more fluorescence dyes.

2. The system of claim 1, wherein the heating element includes at least one of:
a Peltier device;
a heating block;
a thermal electric cooling device;
a hot plate; or
a resistive heater.

3. The system of claim 1, wherein the pathogen detection device is manufactured using three-dimensional (3D) printing.

4. The system of claim 1, wherein the pathogen detection device comprises a resin, the resin comprising:
poly(ethylene glycol) diacrylate (PEGDA);
bis(2,4,6-trimethylbenzoyl)-phenylphosphineoxide (Irgacure 819); and
avobenzone.

5. The system of claim 1, wherein the pathogen detection device further includes one or more microwells, wherein the microwells have dimensions of at about 116 micrometers (μm) by about 116 μm.

6. A device comprising:
a pathogen detection device configured to:
receive, at an inlet of the pathogen detection device, a solution containing at least one of DNA or RNA;
route the solution to a reactive chamber of the pathogen detection device;
heat, via a heating element, the solution;
receive, at the inlet of the pathogen detection device, at least one of a DNA or RNA probe, the probe containing one or more fluorescence dyes;
excite, via an excitation source of an imaging system of the pathogen detection system, the solution and the probe; and
detect, via a fluorescence detection system of the pathogen detection device, an emission of the one or more fluorescence dyes.

7. The device of claim 6, wherein the heating element includes at least one of:
a Peltier device;
a heating block;
a thermal electric cooling device;
a hot plate; or
a resistive heater.

8. The device of claim 6, wherein the pathogen detection device is manufactured using three-dimensional (3D) printing.

9. The device of claim 6, wherein the pathogen detection device comprises a resin, the resin comprising:
poly(ethylene glycol) diacrylate (PEGDA);
bis(2,4,6-trimethylbenzoyl)-phenylphosphineoxide (Irgacure 819); and
avobenzone.

10. The device of claim 6, wherein the pathogen detection device further includes one or more microwells, wherein the microwells have dimensions of at about 116 micrometers (μm) by about 116 μm.

11. A composition comprising:
(a) a nucleic acid having the sequence as set forth in SEQ ID NOs: 1, 3, 4, 5, 6, or 7 or a sequence having at least 95% sequence identity to the sequence as set forth in SEQ ID NO: 1, 3, 4, 5, 6, or 7, linked to
(b) a nucleic acid having the sequence that is the reverse complement to the sequence as set forth in SEQ ID NO: 2 or the reverse complement of a sequence having at least 85% sequence identity to the sequence as set forth in SEQ ID NO: 2; or
(c) a nucleic acid having the sequence as set forth in SEQ ID NOs: 21, 22, 23, 24, 25, 26, 28, 29, 30, 31, 32, or 33, or a sequence having at least 85% sequence identity to the sequence as set forth in SEQ ID NOs: 21, 22, 23, 24, 25, 26, 28, 29, 30, 31, 32, or 33, linked to
(d) a nucleic acid having the sequence that is the reverse complement to the sequence as set forth in SEQ ID NOs: 15, 16, or 27 or the reverse complement of a sequence having at least 85% sequence identity to the sequence as set forth in SEQ ID NOs: 15, 16, or 27;
wherein the sequences of (a) and (b), or (c) and (d) hybridize at about 90° C. and are linked by forming a double helix structure and wherein the nucleic acid of (a) is linked to a fluorophore and located far from the quencher thereby resulting in a fluorescence signal.

12. The composition of claim 11, wherein the nucleic acid of (a) has at least 95% sequence identity to the sequence as set forth in SEQ ID NO: 1, 3, 4, 5, 6, or 7 and the nucleic acid of (b) has at least 99% sequence identity to the reverse complement of the sequence as set forth in SEQ ID NO: 2; or the nucleic acid of (c) has at least 95% sequence identity to the sequence as set forth in SEQ ID NOs: 21, 22, 23, 24, 25, 26, 28, 29, 30, 31, 32, or 33, and the nucleic acid of (d) has at least 95% sequence identity to the reverse complement of the sequence as set forth in SEQ ID NOs: 15, 16, or 27.

13. The composition of claim 11, wherein the nucleic acid of (a) has the sequence as set forth in SEQ ID NO: 1, 3, 4, 5, 6, or 7 and the nucleic acid of (b) has the reverse complement of the sequence as set forth in SEQ ID NO: 2; or the nucleic acid of (c) has the sequence as set forth in SEQ ID NOs: 21, 22, 23, 24, 25, 26, 28, 29, 30, 31, 32, or 33, and the nucleic acid of (d) has the reverse complement of the sequence as set forth in SEQ ID NOs: 15, 16, or 27.

14. The composition of claim 11, wherein nucleic acid forming the loop structure has the sequence as set forth in SEQ ID NOs: 1, 3, 4, 5, 6, 7, 21, 22, 23, 24, 25, 26, 28, 29, 30, 31, 32, or 33, or a sequence having at least 95% sequence identity to the sequence as set forth in SEQ ID NOs: 1, 3, 4, 5, 6, 7, 21, 22, 23, 24, 25, 26, 28, 29, 30, 31, 32, or 33.

15. The composition of claim 11, wherein nucleic acid forming the loop structure has at least 99% sequence identity to the sequence as set forth in SEQ ID NOs: 1, 3, 4, 5, 6, 7, 21, 22, 23, 24, 25, 26, 28, 29, 30, 31, 32, or 33.

16. The composition of claim 11, wherein the bacterial RNA or DNA is derived from streptococcus, Staphylococcus aureus, methicillin-resistant Staphylococcus aureus (MRSA), coliform bacteria, Escherichia coli (E. Coli), Salmonella, Shigella, Clostridium difficile (C. diff), tuberculosis, anthrax, mycoplasma, chlamydophila, legionella, Bordetella pertussis, Clostridium tetani, Borrelia burgdorferi, Borrelia mayonii, Clostridium botulinum, Vibrio cholera, Neisseria meningitides, Haemophilus influenza, Listeria monocytogenes, Treponema pallidum, gardnerelaa vaginalis, or Neisseria gonorrhoeae to name a few.

17. The composition of claim 11, wherein the fungal RNA or DNA encodes Candida albicans, lactobacillus, microsporum canis, tinea pedis, Candida, Aspergillus, Histoplasma, Cryptococcus neoformans (C. neoformans), Blastomyces, or Coccidioides.

18. The composition of claim 11, wherein the parasitic RNA or DNA encodes protozoa, helminths, ectoparasites that cause diseases such as malaria, toxoplasmosis, trichomoniasis, giardiasis, tapeworm, roundworm, lice, scabies, leishmaniasis, or river blindness.

19. The composition of claim 11 wherein the fluorophore or quencher may be a derivative of cyanine (tetramethylindo(di)-carbocyanines), fluorescein amidites (FAM), hexachloro-fluorescein (HEX), carboxyrhodamine (ROX), rhodamine, tetrachlorofluorescein, and the quencher may be a derivative of dimethylaminoazobenzenesulfonic acid (Dabsyl), IRDye (C₅₃H₆₂C|N₄Na₃O₁₆S₄), Iowa Black, Black Hole Quencher.

20. The composition of claim 11, wherein the RNA or DNA is a PCR product that may be the complement of the previously mentioned pathogens in claims 11-20.


SEQUENCE LISTING (33 sequences)

SEQ ID NO: 1 - DNA, 36 bp, synthetic construct
  source 1..36: mol_type = other DNA
gcgggcgaaaagtagatcttcaataaatgagcccgc

SEQ ID NO: 2 - DNA, 99 bp, Severe acute respiratory syndrome-related coronavirus
  source 1..99: mol_type = genomic DNA
acaaatattaccagatccatcaaaaccaagcaagaggtcatttattgaagatctacttttcaacaaagtgacacttgcagatgctggctcatcaaacaa

SEQ ID NO: 3 - DNA, 36 bp, synthetic construct
  source 1..36: mol_type = other DNA
gcgggctcatttattgaagatctacttttcgcccgc

SEQ ID NO: 4 - DNA, 36 bp, synthetic construct
  source 1..36: mol_type = other DNA
cgcccgcttttcatctagaagttatttactcgggcg

SEQ ID NO: 5 - RNA, 36 nt, synthetic construct
  source 1..36: mol_type = other RNA
gcgggcgaaaagtagatcttcaataaatgagcccgc

SEQ ID NO: 6 - RNA, 36 nt, synthetic construct
  source 1..36: mol_type = other RNA
gcgggctcatttattgaagatctacttttcgcccgc

SEQ ID NO: 7 - RNA, 36 nt, synthetic construct
  source 1..36: mol_type = other RNA
cgcccgcttttcatctagaagttatttactcgggcg

SEQ ID NO: 8 - protein, 32 aa, synthetic construct
VGFPVTPQVPLRPMTYKAAVDLSHFLKEKGGL

SEQ ID NO: 9 - protein, 30 aa, synthetic construct
HTQGYFPDWQNYTPGPGVRYPLTFGWLYKL

SEQ ID NO: 10 - protein, 19 aa, Human immunodeficiency virus 1
EKIRLRPGGKKKYKLKHIV

SEQ ID NO: 11 - protein, 32 aa, Human immunodeficiency virus 1
NPPIPVGEIYKRWIILGLNKIVRMYSPTSILD

SEQ ID NO: 12 - protein, 31 aa, synthetic construct
AIFQSSMTKILEPFRKQNPDIVIYQYMDDLY

SEQ ID NO: 13 - protein, 20 aa, synthetic construct
NANPNANPNANPNANPNANP

SEQ ID NO: 14 - protein, 81 aa, synthetic construct
AFTFTKIPAETLHTVTEVQYAGTDGPCKVPAQMAVDMQTLTPVGRLITANPVITEGTENSKMMLELDPPFGDSYIVIGVGE

SEQ ID NO: 15 - RNA, 85 nt, Severe acute respiratory syndrome-related coronavirus
  source 1..85: mol_type = genomic RNA
agtcagtgtgttaatcttacaaccagaactcaattaccccctgcatacactaattctttcacacgtggtgtttattaccctgaca

SEQ ID NO: 16 - DNA, 85 bp, Severe acute respiratory syndrome-related coronavirus
  source 1..85: mol_type = genomic DNA
agtcagtgtgttaatcttacaaccagaactcaattaccccctgcatacactaattctttcacacgtggtgtttattaccctgaca

SEQ ID NO: 17 - DNA, 24 bp, synthetic construct
  source 1..24: mol_type = other DNA
agtcagtgtgttaatcttacaacc

SEQ ID NO: 18 - DNA, 22 bp, synthetic construct
  source 1..22: mol_type = other DNA
tgtcagggtaataaacaccacg

SEQ ID NO: 19 - DNA, 24 bp, synthetic construct
  source 1..24: mol_type = other DNA
ggttgtaagattaacacactgact

SEQ ID NO: 20 - DNA, 22 bp, synthetic construct
  source 1..22: mol_type = other DNA
cgtggtgtttattaccctgaca

SEQ ID NO: 21 - DNA, 46 bp, synthetic construct
  source 1..46: mol_type = other DNA
gcgccctgtcagggtaataaacaccacgtgtgaaagaattgggcgc

SEQ ID NO: 22 - DNA, 12 bp, synthetic construct
  source 1..12: mol_type = other DNA
tgcagggggtaa

SEQ ID NO: 23 - DNA, 40 bp, synthetic construct
  source 1..40: mol_type = other DNA
cgcccgttctggttgtaagattaacacactgactcgggcg

SEQ ID NO: 24 - DNA, 46 bp, synthetic construct
  source 1..46: mol_type = other DNA
gcgcccaattctttcacacgtggtgtttattaccctgacagggcgc

SEQ ID NO: 25 - DNA, 12 bp, synthetic construct
  source 1..12: mol_type = other DNA
ttaccccctgca

SEQ ID NO: 26 - DNA, 40 bp, synthetic construct
  source 1..40: mol_type = other DNA
cgcccgagtcagtgtgttaatcttacaaccagaacgggcg

SEQ ID NO: 27 - DNA, 85 bp, Severe acute respiratory syndrome-related coronavirus
  source 1..85: mol_type = genomic DNA
tgtcagggtaataaacaccacgtgtgaaagaattagtgtatgcagggggtaattgagttctggttgtaagattaacacactgact

SEQ ID NO: 28 - RNA, 46 nt, synthetic construct
  source 1..46: mol_type = other RNA
gcgccctgtcagggtaataaacaccacgtgtgaaagaattgggcgc

SEQ ID NO: 29 - RNA, 12 nt, synthetic construct
  source 1..12: mol_type = other RNA
tgcagggggtaa

SEQ ID NO: 30 - RNA, 40 nt, synthetic construct
  source 1..40: mol_type = other RNA
cgcccgttctggttgtaagattaacacactgactcgggcg

SEQ ID NO: 31 - RNA, 46 nt, synthetic construct
  source 1..46: mol_type = other RNA
cgcgggacagtcccattatttgtggtgcacactttcttaacccgcg

SEQ ID NO: 32 - RNA, 12 nt, synthetic construct
  source 1..12: mol_type = other RNA
ttaccccctgca

SEQ ID NO: 33 - RNA, 40 nt, synthetic construct
  source 1..40: mol_type = other RNA
cgcccgagtcagtgtgttaatcttacaaccagaacgggcg